(19) United States
(12) Patent Application Publication
(10) Pub. No.: US 2009/0160075 A1
(43) Pub. Date: Jun. 25, 2009

(54) METHODS FOR FABRICATING CUSTOMIZED INTRAOCULAR LENSES

(76) Inventors: Michael J. Simpson, Arlington, TX (US); Daniel R. Carson, Fort Worth, TX (US); Kamal K. Das, Arlington, TX (US)

Correspondence Address: ALCON, IP LEGAL, TB4-8, 6201 SOUTH FREEWAY, FORT WORTH, TX, 76134, US

(21) Appl. No.: 12/337,192

(22) Filed: Dec. 17, 2008

Related U.S. Application Data
(60) Provisional application No. 61/016,241, filed on Dec. 21, 2007.

(51) Int. Cl. B29D 11/00; A61F 9/007

(52) U.S. Cl. 264/1.37; 606/107

(57) ABSTRACT

In one aspect, the present invention provides methods for custom fabrication of IOLs. In some embodiments, such methods call for measuring one or more aberrations of a patient's eye, and determining the profile of at least one surface of an IOL that would ameliorate, and control those aberrations. The surface profile can then be imparted to a surface of a starting lens (or a lens blank) via ablation, e.g., by utilizing an excimer laser beam. In some other embodiments, the measured aberrations can be utilized to determine the profile of at least one surface of a wafer mold. A wafer mold having that surface profile can then be fabricated, e.g., by ablating a slab or an existing wafer of appropriate material, and the mold can be used to fabricate an IOL suitable for implantation in the patient's eye.

### RELATED APPLICATIONS

This application claims priority to provisional application Ser. No. 61/016,241, filed on Dec. 21, 2007, the contents of which are incorporated herein by reference.

### BACKGROUND

The present invention relates generally to methods of fabricating ophthalmic lenses, and more particularly, to methods for custom fabrication of intraocular lenses (IOLs).

Intraocular lenses are routinely implanted in patients' eyes during cataract surgery to replace the natural crystalline lens. The optical power of the IOL is typically specified so that the eye is close to emmetropia, or perhaps slightly myopic, after surgery. However, a patient's eye can have its own unique optical characteristics including some degree of optical aberration. The optical properties of conventional IOLs are not matched to the optical needs of an eye of a particular patient. Rather, such IOLs are generally specified by their optical power, and not by the image quality that they might provide. In some instances, toric IOLs are also available for correcting astigmatism. However, such lenses are typically available for a small range of astigmatic corrections. Moreover, they do not address higher order imaging aberrations that can be present in a patient's eye.

Accordingly, there is a need for improved designs for IOLs and the like that can provide enhanced vision correction as well as better methods for fabricating optical devices suitable for such vision corrections.

### SUMMARY

In one aspect, the present invention provides a method of fabricating an intraocular lens (IOL), which comprises measuring one or more aberrations of a patient's eye, determining at least one surface profile of a mold wafer based on those measurements, ablating at least one surface of a mold wafer to impart that profile to the surface, and utilizing the mold to fabricate an IOL, e.g., via a casting process, suitable for implantation in the patient's eye. A pair of mold wafers is typically used to fabricate a single lens, after which they are discarded, and they can be formed of a variety of materials, such as polypropylene.

In a related aspect, the ablation parameters, e.g., fluence, for ablating the mold wafer can be determined based on the properties of the material from which the mold wafer is made. By way of example, when utilizing a mold wafer formed of polypropylene, a radiation fluence greater than about 100 mJ/cm², e.g., in a range of about 100 mJ/cm² to about 800 mJ/cm² can be employed.

In another aspect, a method for fabricating an optical device such as an IOL is disclosed, which comprises measuring one or more aberrations of a patient's eye, determining one or more surface profiles for an optical device, and ablating a substrate formed from a polymeric material so as to fabricate a device having said surface profiles. The substrate can be a starting lens (or a lens blank) at least one surface of which can be ablated to customize it for implantation in the patient's eye.

In a related aspect, the substrate (e.g., a lens blank) can be formed of a polymeric material such as Acrysof®, hydrogel, or silicone. One or more ablative parameters can be selected based on the material properties of the substrate. For example, when the substrate is formed of Acrysof®, the fluence of the ablative radiation can be in a range of about 10 mJ/cm² to about 600 mJ/cm², and preferably in a range of about 200 mJ/cm² to about 500 mJ/cm².

Further understanding of the invention can be obtained by reference to following detailed description in conjunction with the associated drawings, which are described briefly below.

### BRIEF DESCRIPTION OF THE DRAWINGS

FIG. 1 is a flow chart depicting various steps for practicing some embodiments of methods according to the invention for fabricating an IOL,

FIG. 2 is a schematic cross-sectional view of a mold wafer having a concave surface whose profile can be adjusted via ablation to obtain a customized mold wafer for fabricating a IOL suitable for a particular patient,

FIG. 3 schematically depicts an excimer ablation system suitable for use in the practice of various methods of the invention,

FIG. 4 is a schematic cross-sectional view of a starting IOL retained in one of the mold wafers initially used to fabricate it with its anterior surface exposed for customizing ablation,

FIG. 5 schematically depicts a slab of lens material that can be ablated to determine its fundamental ablation characteristics,

FIG. 6 is a schematic layout of ablation spots applied to a polypropylene slab mold wafer in an illustrative experiment,

FIG. 7A presents data for polypropylene corresponding to ablation depth per pulse as a function of various pulse numbers for five different fluences,

FIG. 7B presents data for polypropylene corresponding to ablation depth per pulse as a function of fluence for different pulse numbers,

FIG. 8 presents comparative ablation rate data for Acrysof®, Acrysof Natural and PMMA as a function of fluence, and

FIG. 9 is a graph depicting actual dioptric change generated in an Acrysof® wafer via ablation versus a respective nominal (attempted) change,

### DETAILED DESCRIPTION

The present invention relates generally to methods for custom fabrication of ophthalmic lenses. Although the embodiments discussed below are generally directed to fabrication of IOLs, the teachings of the invention can be applied to fabrication of other ophthalmic lenses, such as pseudophakic lenses, intrastromal lenses, and contact lenses. Further, the term intraocular lens and its abbreviation “IOL” are used herein interchangeably to describe lenses that can be implanted into the interior of an eye to either replace the eye's natural crystalline lens or to otherwise augment vision regardless of whether or not the natural lens is removed.

In some embodiments, a customized IOL can be fabricated by selectively ablating, e.g., via an excimer laser beam, a surface of a lens (or a lens blank) formed of a flexible polymeric material, such as an acrylic material, so as to adjust the surface profile such that the lens would accommodate the unique optical needs of a patient's eye in which the lens would be implanted. By way of example, in some embodiments, the lens (or the lens blank) can be formed of a cross-linked copolymer of 2-phenylethyl acrylate and 2-phenylethyl methacrylate, commonly known as Acrysof®. It was discovered that the Acrysof® material exhibits an incubation phenomenon when exposed to ablative radiation. Incubation has been observed for other materials, where the amount of material removed by initial laser pulses differs from the amount of material removed by later pulses, but this had not previously been found for Acrysof®. In addition, however, it was found that the amount of material removed via an ablative pulse from a location of an Acrysof® substrate varies with the both the local fluence and the previous history of ablative radiation fluences at that location. As discussed in more detail below, the incubation characteristic that is defined for constant fluence across a region of the surface, must be modified to reflect the effect that cumulative ablations have at a single point if the local fluence changes from shot to shot. This is important where a scanning laser spot is used to ablate an optical quality surface, and it should be taken into account when selecting ablation parameters, e.g., fluence, so as to produce an optically smooth surface. By way of example, in some embodiments, a surface of a lens (or a lens blank) is ablated, then the surface profile is measured, and the surface is ablated again, if needed, to correct surface profile errors, if any, that were observed. This iterative process can be repeated as many times as needed to arrive at a surface profile with surface irregularities, if any, that are below a desired threshold.

It was also discovered that it is advantageous to firmly hold a lens's position relative to an ablative laser beam. By way of example, in some embodiments this can achieved by retaining the lens in one of the two wafers between which the lens was originally cast and removing the other wafer to expose a lens surface to be ablated. In some other embodiments, a lens can be fixated relative to an ablative laser beam via suitable fixturing.

It was also discovered that if the ablation energy is too high, a lens can experience surface cracking when the lens is folded. Hence, as discussed further below, the ablation energy should preferably be selected to avoid such surface cracking.

In some other embodiments, rather than ablating a lens surface to customize the lens for use in a patient's eye, a surface of a mold wafer can be ablated, based on measured aberrations of the patient's eye, so as to generate a surface profile suitable for fabricating a lens that is customized for that patient. The wafer can be used, e.g., in conjunction with another wafer, to fabricate the lens, e.g., via a casting process. Hence, in some cases, two wafers, one of which is customized for a particular patient, can be utilized to fabricate the lens. The customized wafer can be disposable to be replaced with a different one suitable for fabricating a lens for another patient. The mold wafer can be formed, e.g., from a suitable soft polymeric material such as polypropylene. It was discovered that polypropylene also exhibits an incubation phenomenon that needs to be taken in account when ablating a polypropylene wafer.

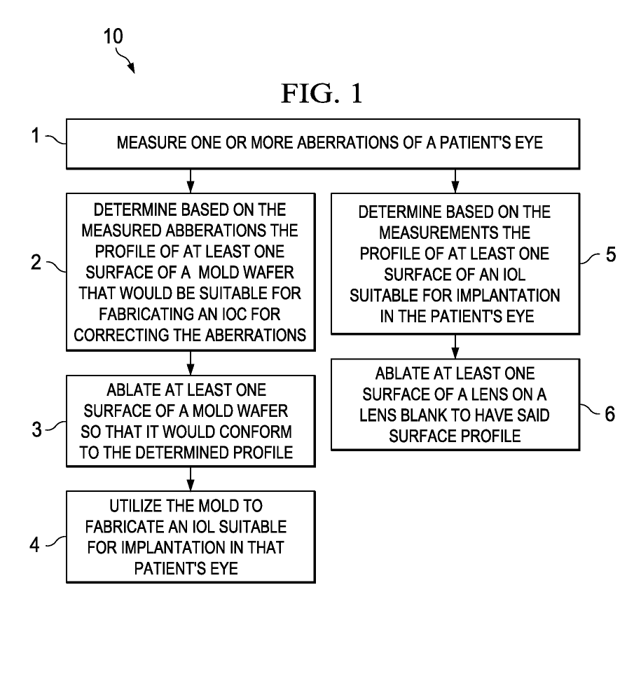

With reference to a flow chart 10 of FIG. 1, in one embodiment of a method of the invention for fabricating an intraocular lens, one or more aberrations of a patient's eye are measured (step 1). Such aberrations can comprise a plurality of symmetric and/or asymmetric aberrations, including without limitation, astigmatism, coma, spherical aberration, trefoil, etc. The measurement of the aberrations can be done for pseudophakic or phakic implants. In some cases, corneal aberration information can be used for the former, and total eye aberration information can be used for the latter. A variety of techniques and instruments can be employed to measure the aberrations. By way of example, a Hartmann-Shack wavefront sensor can be utilized to measure the aberration of the eye. In such a sensor, the light exiting the eye in response to illumination of a retinal spot by focused light is directed to an array of lenslets, each of which generates an image of the light incident thereon on a detector, e.g., a CCD camera. These images can be analyzed in a manner known in the art to reconstruct the returning wavefront, and hence determine one or more aberrations of the eye. In many embodiments, the reconstructed wavefront can be represented as a sum of a plurality of Zernike polynomials, which constitute a set of orthogonal polynomials on a unit circle. The coefficients of the polynomials correspond to different aberration types. By way of example, the reconstructed wavefront (Z(ρ, θ)) can be represented in the following manner:

$$z ( ρ , θ ) = {\sum }_{i = 1}^{15} {α}_{i} {Z}_{i} ,$$

wherein,
ρ and θ represent, respectively, the normalized radius and azimuth angle,
Zᵢ represents a Zernike polynomial of order i, and
αᵢ represents a Zernike coefficient of order i,

The aberration information can be utilized for custom fabrication of a mold wafer, which can in turn be employed to fabricate a corresponding IOL for implantation in the patient's eye. Alternatively, the aberration information can be employed to customize an IOL (e.g., via ablation of one or more surfaces of an IOL lens or a lens blank) for the patient.

For example, with continued reference to the flow chart 10, in a subsequent step (2), at least one surface profile of a mold wafer, e.g., a polymeric mold, suitable for generating an IOL whose implantation in that patient's eye would control those aberrations is determined. Although the mold can generally be formed of any suitable material, in many embodiments, it can be formed of a polymeric material, such as polypropylene.

Once the desired surface profile of the mold is determined, at least one surface of a mold wafer can be ablated, e.g., via an excimer laser, such that it would conform to that surface profile (step 3). The mold can then be utilized in a manner known in the art to fabricate an IOL having the desired surface profile (step 4). By way of example, in many embodiments, the mold can be employed, e.g., after standard cleaning, to cast an IOL from a biocompatible polymeric material, such as phenylethylacrylatephenylethylmethacrylate, known as Acrysof®. In this manner, a personalized IOL can be fabricated that can optimize the optical performance of the patient's eye after IOL implantation.

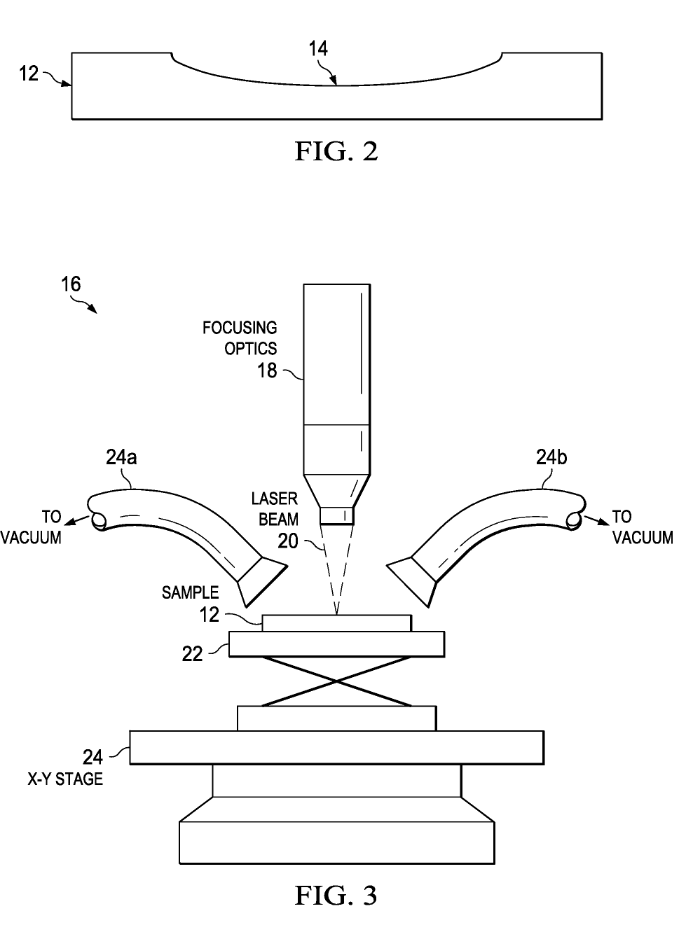

By way of further illustration, FIG. 2 schematically depicts a starting polymeric mold 12 having a concave surface 14 representing a rotationally symmetric surface having a selected radius of curvature. The starting surface 14 of the mold 12 can be further shaped via ablation to arrive at a mold surface suitable for correcting aberrations of an eye of a particular patient. For example, the ablation can impart a profile to the surface 14 that is suitable for generating an IOL that provides not only a desired refractive power but can also correct one or more higher-order aberrations of the patient's eye, such as spherical aberration or trefoil. One skilled in the art will appreciate that such techniques can also be used to make an IOL that corrects other types of aberrations, such as astigmatism.

Such ablation of the mold 12 can be achieved, for example, by utilizing an excimer laser system. By way of example, FIG. 3 schematically depicts such a system 16 that includes an excimer laser 18, and associated focusing optics, providing a laser beam 20, e.g., at a wavelength of about 193 nm. A variety of excimer lasers can be utilized in the practice of the invention. Such lasers can provide various beam cross-sectional profiles, e.g., flat-top or gaussian. By way of example, an excimer laser system marketed by Resonetics, Inc. of Nashua, N.H., USA operating at 193 nm and providing a flat-top laser beam can be employed. Alternatively, an excimer laser marketed by Alcon Laboratories, Inc. of Fort Worth, Tex., USA under trade designation LADARVision operating at 193 can be utilized.

With continued reference to FIG. 3, the mold 12 can be placed on a sample holder 22 in the path of the laser beam such that its surface 14 would be exposed to the beam. In some other embodiments in which a lens or a lens bank is ablated, the sample holder can preferably provide positional fixation of the lens so as to prevent unwanted movements of the lens, e.g., as a result of the impact of a plurality of ablative radiation pulses. The exemplary system 16 further includes a plurality of vacuum lines 24a and 24b that facilitate the removal of polymeric debris generated as a result of laser ablation of the mold's surface. In this case, the holder 22 is disposed over an X-Y translation stage 24 that can move the mold in two dimensions according to a preprogrammed pattern to cause ablation of selected portions of the mold's surface 12, thereby generating a desired mold profile. In alternative embodiments, rather than moving the mold relative to the laser beam, the beam itself can be moved over the mold's surface according to a preprogrammed pattern to cause selected ablation of the surface. Such excimer laser systems are commercially available, such as the aforementioned LADARVision excimer laser, and are routinely employed for corneal laser correction. The optical surface will typically have a preferential orientation, and the wafer can be centered and oriented using appropriate fixturing.

A plurality of ablation patterns (e.g., a multi-spiral pattern) can be utilized to arrive at a desired mold surface profile. In some patterns, two or more adjacent ablation regions can overlap to avoid the generation of ridges between those regions, thereby providing a smoother final surface. The ablation patterns suitable for a variety of optical aberration corrections are well-known in corneal laser correction methods, and can be readily adapted in the practice of various embodiment of the invention.

The radiation fluence for ablating the mold wafer 12 can be selected based on the material from which the mold is formed. By way of example, in some embodiments in which the mold is formed of polypropylene, the fluence for ablating the mold is selected to be greater than about 100 mJ/cm². For example, such a fluence can be in a range of about 100 mJ/cm² to about 800 mJ/cm².

Although in the above exemplary embodiment, the starting mold surface 12 has a concave profile, in other embodiments, the starting mold surface to be ablated can be flat, or it can have a convex surface. For example, the starting mold can have flat surfaces. At least one of the mold surfaces can be ablated, e.g., in a manner discussed above, to provide a mold surface having a suitable profile for shaping the respective surface of an IOL that is customized for a particular patient.

In some cases, an anterior surface of an IOL can be shaped by one mold wafer and its posterior surface can be shaped by another. At least one of those wafers can include a surface having a profile achieved by ablation based on the needs of a particular patient. The two wafers can be employed in a manner known in the art to fabricate an IOL from a suitable biocompatible material. For example, the wafers can be formed of polypropylene and can be employed to fabricate an IOL from phenylethyl acrylate-phenylethyl methacrylate polymeric material, which is known as Acrysof®, via a casting process.

Referring again to the flow chart 10 of FIG. 1, in alternative embodiments, rather than selectively ablating a mold to achieve one suitable for custom fabrication of an IOL, one or more optical surfaces of a starting lens can be ablated to impart a custom profile to those surfaces so as to form an IOL from the lens blank that can accommodate the visual needs of a particular patient. More specifically, the measured aberration(s) can be utilized to determine the profile of at least one surface of an IOL to be fabricated (step 5) that would facilitate controlling the aberrations. Subsequently, at least one surface of a lens (or a lens blank) can be ablated so as to impart the profile to that surface (step 6).

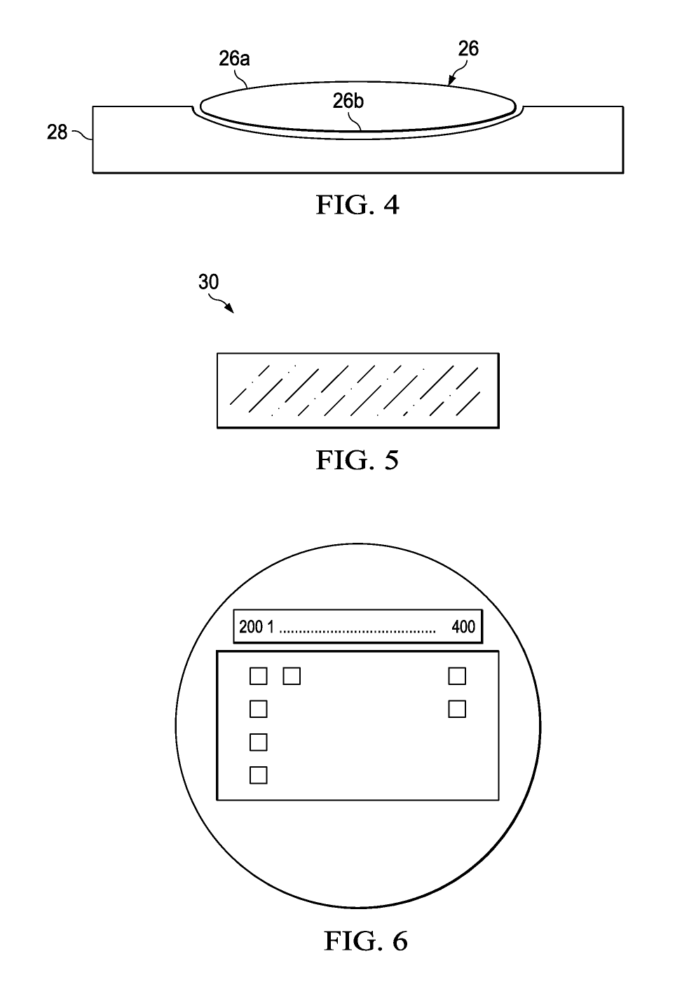

By way of example, FIG. 4 schematically depicts such a starting lens blank 26 formed of Acrysof® that includes an anterior surface 26a and a posterior surface 26b, one or both of which can be shaped via laser ablation to generate an IOL suitable for correcting visual needs of a patient. In this case, the starting lens 26 includes curved surfaces that provide the lens with a nominal optical power, which can be adjusted to be customized for a particular patient. In addition, the surface can be further shaped to provide correction for one or more higher order aberrations of the patient's eye.

With continued reference to FIG. 4, in this example, the anterior surface of the lens 26 can be ablated, e.g., via an excimer laser, while the lens remains in one of the two mold wafers (mold 28) in which it was originally cast. In this example, the starting lens is assumed to be formed of Acrysof®. It was found that Acrysof® exhibits an incubation phenomenon when exposed to ablative pulses. In other words, the amount of material removed can vary based on the previous history of ablation and illumination. For example, in some experiments, initial ablative pulses were found to remove more material than later pulses having the same energy. Further, it was found that when an Acrysof® material is exposed to a scanning ablative laser spot having a variable intensity profile, the amount of material removed can be affected by the intensity variation across the spot in a manner not expected from constant fluence experiments. For example, when exposing an Acrysof® surface to a gaussian beam, the brighter central region of the beam causes ablation at a higher rate than the fainter peripheral beam. However, the local removal rate can be different than that expected from data corresponding to ablating the surface with a rectangular beam having a comparable fluence. It was also found that ablating an Acrysof lens surface at too high an ablation energy, the resultant lens can exhibit microcracks upon folding and unfolding.

The above factors should be taken into account when ablating an Acrysof® lens surface, such as the lens surface 26a, e.g., via an excimer laser operating at a wavelength of 193 nm. By way of example, in many embodiments in which an Acrysof® lens (or a lens blank) is ablated to customize the lens for a particular patient, a radiation fluence in a range of about 200 mJ/cm² to about 500 mJ/cm² can be employed. The choice of the fluence can be affected by the intensity profile of the radiation beam. For example, for a gaussian laser beam at a wavelength of 193 nm, the radiation fluence for ablating an Acrysof® lens (or a lens blank) can be in a range of about 10 mJ/cm² to about 600 mJ/cm², and preferably in a range of about 200 mJ/cm² to about 500 mJ/cm². In some embodiments in which an excimer laser beam having a rectangular intensity profile is utilized to ablate an Acrysof® lens (or lens blank), the radiation fluence can be in a range of about 200 mJ/cm² to about 500 mJ/cm².

The polymeric material from which the starting lens or lens blank is formed is not limited to Acrysof®, and generally can be any suitable biocompatible polymeric material. Some other examples of such polymeric materials include, without limitation, hydrogel and silicone. By way of further examples, U.S. Pat. No. 6,416,550, which is herein incorporated by reference, discloses materials suitable for forming the IOL. The material properties of such materials, e.g., volume of material removed per ablation pulse, should be taken into account in calculating an ablation pattern. In some embodiments in which the lens is formed of a hydrophobic polymeric material, the fluence of ablative radiation can be in a range of about 10 mJ/cm² to about 1000 mJ/cm².

In the above case, the anterior and the posterior surfaces of the lens 26 are curved such that the starting lens would provide a nominal optical power, thereby minimizing the amount of material that needs to be removed in order to customize the lens for a particular patient. In some other embodiments, a lens blank having flat surfaces can be ablated to provide a customized IOL for a patient. Similar to the previous embodiments, the aberrations of a patient's eye can be measured and one or more surfaces of the lens blank can be ablated to provide an IOL that can control those aberrations when implanted in that patient's eye. By way of example, such ablation of the lens blank's surface(s) can impart a desired optical power to the resultant lens as well as, if needed, shape its surface(s) so as to correct one or more higher aberrations of the eye.

In some cases, following ablation of one or more surface(s) of a lens or a lens blank, the profiles of those surface(s) can be measured, and those surface(s) can be subjected to another ablation, if needed, so as to reduce surface profile errors. This process can be repeated as many times as needed to arrive at a smooth lens surface, e.g., until the surface profile exhibit surface irregularities below a selected threshold (e.g., defined as P-V or RMS).

In some cases, a pattern of corrective ablative pulses can be applied to a surface of a lens (or a lens blank), or that of a mold wafer, after exposing the surface to shaping ablating pulses (pulses designed to impart a selected profile to the surface) to reduce surface irregularities based on a pre-determined pulse pattern. Such a pulse pattern can be determined by utilizing a substrate formed of the same material and having a comparable surface by exposing that surface to a similar pattern of shaping ablative pulses and subsequently measuring irregularities in the surface profile. A corrective pattern of ablative pulses can then be determined so as to reduce those irregularities. Once this corrective pattern is determined, it can be applied to other comparable substrates that were subjected to the same pattern of shaping ablation pulses for shaping/adjusting their profiles without a need to measure the irregularities for each individual substrate.

Moreover, in some cases, the pattern of residual surface error can be similar for similar types of ablations. As such, a corrective pattern of ablation determined for one substrate can be applied to other substrates that are subject to similar—and not necessarily identical—ablation patterns.

In some cases, one or more characteristics of multiple ablations using a particular spot profile can be determined, and then used, e.g., via modeling calculations, to determine an optimal ablative shot pattern for a scanning spot.

In some cases, the ablation of a polymeric surface, e.g., an Acrysof® surface, can be achieved by applying multiple sets of ablative pulses to the surface with a quiescent period (i.e., a period during which no pulses are applied) between any two ablative sets. Such quiescent periods allow the material recover between the ablation sessions (between different ablation sets), as well as allows for plume removal, if needed. For example, a scanning ablation spot can be moved in a pattern on the substrate surface to generate a pattern of ablation. This can be followed by a quiescent period. Then, the scanning ablation spot can be moved on the substrate again to cause ablation. This process can be repeated until a desired profile of the surface is achieved.

In some cases a lens surface can be ablated for customization to a patient's need before the lens is removed from one of the two mold wafers between which it was initially cast (See, e.g., FIG. 4). This provides a number of advantages. For example, the lens can be securely attached to the wafer and it can be accurately positioned relative to an ablative scanning laser beam. The ablated material can be removed by utilizing standard lens washing techniques known in the art. The lens can then be extracted from the wafer by employing standard techniques. Other standard processing steps can then be applied, e.g., plasma treatment. In other alternative embodiments, the lens can be ablated later in the fabrication process, even as a finished lens. In some cases, the customizing ablation can even be performed just prior to the implantation of the lens while providing attention to the removal of the ablation products and the maintenance of sterility.

Although in the above embodiments, the various aspects of the invention are discussed with reference to monofocal IOLs, the teachings of the invention can also be applied to multifocal IOLs to customize them for use in patients' eyes. By way of example, such a multifocal IOL can include an anterior surface and a posterior surface. A plurality of diffractive structures can be disposed on the anterior surface of the lens such that the lens would provide not only a far-focus optical power but also a near-focus optical power. By way of example, in such a case, the posterior surface of the lens can be ablated, e.g., in a manner discussed above, so as to customize the lens to the needs of a particular patient.

The teachings of the invention can also be employed to provide fine-tuning of the optical power of standard IOLs. For example, a specified level and orientation of cylindrical power can be provided, or a specified magnitude of asphericity can be added to a lens.

The lens fabrication methods of the invention provide the flexibility of modifying the optical properties of a lens to meet the individual needs of a patient or a surgeon. For example, such a lens can provide a personalized correction for spherical power, cylindrical error, spherical aberration, and higher order aberrations of an individual patient. Further, in many cases, standard methods of lens casting, sterilization and packaging can be utilized.

The following examples are provided to further illustrate various aspects of the invention. It should be understood that the examples are presented only for illustrative purposes and are not intended to necessarily indicate optimal ways of practicing the invention or optimal materials from which the molds or the IOLs can be fabricated. In particular, the described methods may be applied to a number of soft acrylic IOL materials, including AcrySof® materials described in U.S. Pat. Nos. 5,290,892 and 5,693,095 (the latter of which is hereinafter referred to as “AcrySof II”). As will be apparent to one skilled in the art, these materials may be bound with chromophore materials as well, referred to herein as “AcrySof Natural” or AcrySof II Natural.”

### EXAMPLE 1

The fundamental ablation properties of the lens material and the mold wafer material were determined using “slabs” of the material, and corresponding slab wafer molds. FIG. 5 schematically depicts a slab of material. Polypropylene slab molds were ablated by employing an excimer laser operating at 193 nm. Each mold was in the form of a circular disk having a diameter of about 31 mm, with a 1 mm deep, 20 mm×10 mm rectangular depression in the center. The polypropylene molds were not plasma treated. Ten (10) polypropylene slab molds were ablated with various numbers of laser pulses and various fluences. Each sample was covered with a polypropylene disk of the same diameter when not in use to avoid dust and contamination.

A pulsed ultraviolet (UV) excimer from Lambda Physik (Gottingen, Germany) at an emission wavelength of 193 nm and at a pulse repetition rate of 60 Hz was used for ablation. The laser provides a substantially uniform beam profile with an energy variation of about ±5%. A mask was used at the exit plane of the laser to limit the beam. The image of the mask was formed at the surface of the specimen. A summary of some of the experimental parameters is presented below:

Demag: 8.76x

Lens: f=200 mm lens before mask

Assist Gas: Vacuum suction from dual nozzles approximately 5 mm from the target

Fluence: Table X below provides fluence values used to ablate the slabs

Tooling: Substrates were attached to a manual z-stage with Kapton tape. A variable attenuator was mounted between the laser and workstation

Mask: RVA set to about 0.110 inches×0.352 inches

Spot dimensions: Rectangle, about 0.32 mm×1.02 mm

Laser pulse rate: 60 Hz

FIG. 6 shows a schematic layout of a polypropylene slab with the big rectangle inside the circle representing the ablation area. Each small rectangle inside the big rectangle represents an ablation area or spot. Four different rows of ablation spots were utilized, where each row contained 18 ablation spots. The top vertical bar indicates the number of pulses applied to a respective spot in a row for generating an ablation spot. The horizontal pitch between the spots was about 0.9 mm, and the vertical pitch between the spots was about 1.6 mm, for all slabs in this experiment. Ablation spots were laid out in a consistent and well-ordered rectilinear array on each sample. The first spot on each slab was exposed to many pulses (200 pulses) to facilitate measurements after ablation.

Twenty different laser fluences were used to ablate the propylene slab molds. To derive these fluence values, a Molectron™ power detector was used to measure the laser energy at the specimen surface. The fluence was then derived by dividing the measured laser energy by the known ablation area. (Laser output, and thus measured energy, varied by about ±5%. The fluence values can also have some residual error as nominal filtering values can be different than the actual values.)

A Form Talysurf profilometer was employed to measure the ablation depth profiles of the ablated slabs. The profilometer had a height resolution of 10 nm (0.01 microns). The resolution value is smaller than the ablation depths evaluated in these experiments. Custom software was used to determine the depth of each ablated region. The ablation depth per pulse (microns/pulse) at each laser fluence was calculated from the profilometer data. Likewise, the ablation depths for all of the ablated polypropylene slabs were analyzed at all laser fluences.

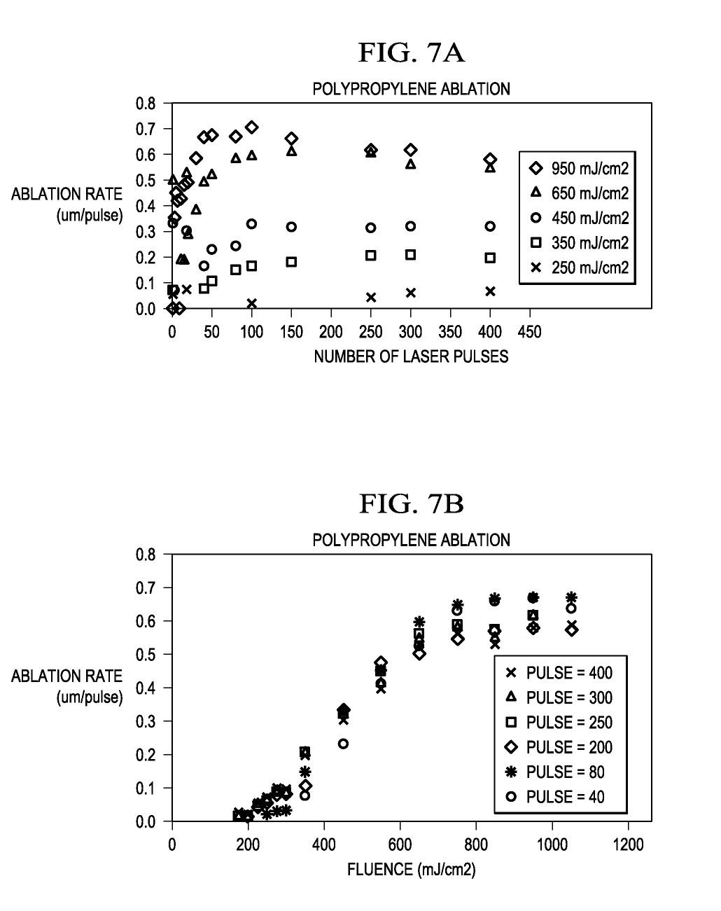

FIG. 7A presents ablation per pulse (μm/pulse) as a function of various laser pulses for five different fluences of 250, 350, 450, 650, and 950 mJ/cm². FIG. 7B presents polypropylene ablation rate data as a function of fluence for different pulse numbers. The data suggest an increase in ablation rate from the initial laser pulse to the laser pulse of 100, which in turn suggests strong “incubation” effects for polypropylene material. The ablation rate does not appear to change for 100 or more pulses when the energy is above saturation. The ablation rate, however, appears to decrease for 100 or more pulses when the energy is below saturation.

### EXAMPLE 2

Slabs of the following three types of lens materials were ablated by employing the aforementioned Lambda Physik (Gottingen, Germany) excimer laser operating at 193 nm at a repetition rate of 60 Hz: Acrysof, Acrysof Natural and PMMA (polymethylmethacrylate). FIG. 3 above shows a schematic layout of the experimental set-up that was employed to conduct the ablation experiments. A pair of vacuum debris removal nozzles was used to suction away ablation by-products and minimize redeposit on the surface. A mask was used at the exit plane of the laser to limit the beam. The image of the mask was formed at the surface of the specimen. An X-Y stage was used for linear motion. A Molectron™ power meter was used to measure laser energy (in mJ) at the sample surface. The measurements of the laser output suggested a laser energy variation of about ±5% or less. The laser fluence (mJ/cm²) was obtained by dividing the energy by the ablation area for each material ablation. The fluence level accuracy was achieved by stabilizing the laser energy at a constant level and using appropriate filter combinations. The laser output (and thus measured energy) varied by about ±5%, and also the nominal filter values utilized to calculate the energy at sample and fluences could be somewhat different that the respective actual values.

The Form Talysurf stylus profilometer was used to obtain surface profile data from the ablated samples. This profilometer has a height resolution of 0.01 microns (10 nm), which is less than the depths of the ablation regions under evaluation, thus ensuring ablation depth measurement accuracy.

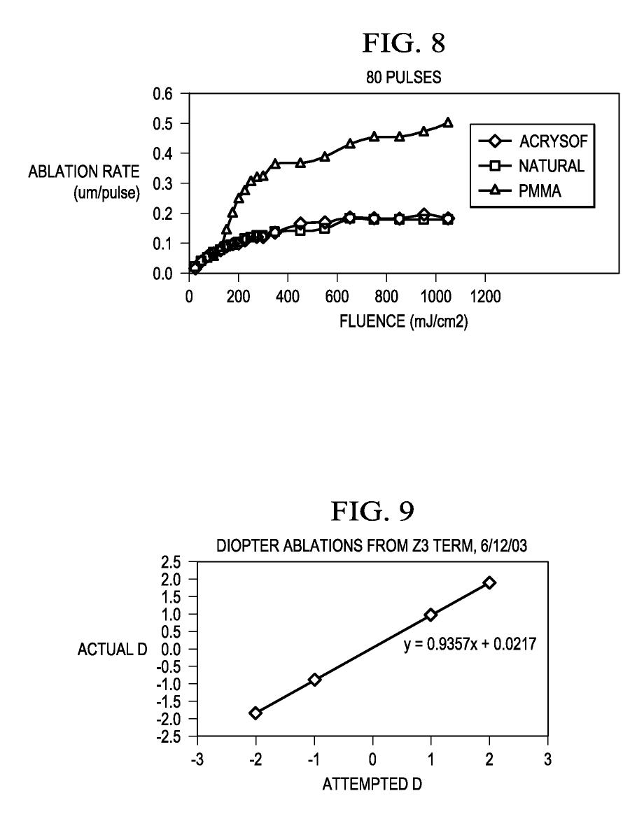

FIG. 8 provides a comparison of ablation rate for 80 laser pulses as a function of fluence for Acrysof®, Acrysof Natural, and PMMA. The data indicates that PMMA requires a higher threshold energy for ablation (about 100 mJ/cm² higher). However, it can be more readily ablated than Acrysof® and Acrysof® Natural at fluences beyond the threshold value. The material removal per pulse is about 0.4 microns/pulse for PMMA as compared to about 0.18 microns/pulse for both Acrysof and Acrysof Natural.

### EXAMPLE 3

The LADARVision® 4000 excimer laser system of Alcon, Inc. (assignee of the present application) was used to both change lens power and to correct small amounts of aberration on lens surfaces formed of AcrySof®. Samples for lens ablations were lens blanks, consisting of Acrysof cast between two polypropylene mold wafers, and then released from one side. These samples were cured but not extracted, and they had larger fabrication errors than normal to provide an opportunity for the correction of aberrations. Most samples had three to six fringes of error across the 6.0 mm diameter of the surface, including some astigmatism.

LADARVision® 4000 is a clinical laser system that is primarily designed to ablate the cornea. Its software incorporates the ablation characteristics of both the cornea and PMMA, which are stored as curves of ablation depth versus laser fluence (in mJ/mm²). The system software also allows the user to specify the beam parameters. The system calculates a correction pattern for the cornea using the theoretical volume of material removed by each pulse of the laser, or volume per shot (VPS). It computes the VPS of corneal material removed by the laser by measuring the size of a spot ablated on a piece of Mylar during a step-up procedure. The system computes the volume of corneal tissue removed by multiplying the VPS by the number of applied shots. Since it is known how much volumetric tissue needs to be removed for each prescription of myopia, hyperopia and astigmatism, the system can simply calculate the number of shots required at each ablation site. For a given laser energy and beam profile, the system's software computes the VPS and the shot pattern needed to remove enough material to obtain the desired surface profile change. The resulting shot pattern can be stored and used to control the laser system.

In order to compute shot patterns for ablating the lens blanks, VPS values for Acrysof were measured by utilizing the LADARVision® laser system. The measurements were made by employing standard Acrysof slabs. A spot pattern file was created for the LADARVision® system to generate multiple shots laid out in a square of four spots, measuring four millimeters on a side. The four locations corresponded to 50, 100, 150, and 200 laser shots, respectively. The pattern was loaded into LADARVision® system and the samples were ablated at 1.35 mJ energy and at a shot repetition rate of 60 Hz. The beam energy was confirmed by employing a Molectron® power meter.

The volume of an ablated spot was determined using an ADE-Phase Shift MicroXAM white light interferometer, which was configured to provide a maximum field of view of about 3.2×2.4 millimeters. The spot was measured to be about 1.6 mm×1.8 mm with a depth of about 14 microns.

For ablating surfaces of the lens blanks, the surfaces were represented by one or more Zernike polynomials. Optical surfaces of a lens are often described by their local sagittal heights, or “sag,” which represents the local distance along an axial direction from a plane through the apex of the lens. By way of example, converting a radius of curvature of a surface to an equivalent representation as a Zernike value can be achieved in the following manner in the paraxial regime:

$${Z}_{3} = \frac{{r}_{max}^{2}}{4 {R}_{C}}$$

wherein,
Z₃ represents the Zernike term corresponding to power (3^{rd} term here)

rₘₐₓ represents maximum radius of the surface (semi-diameter), and

R_{C} represents the radius of curvature of the surface.

There are several different definitions for Zernike polynomials, and the numbering scheme used here designated Z₃ as the power term. For a +1 D ablation, a Z₃ term of 0.0034834 was employed. The Z₃ term was doubled to 0.0069668 for +2 D ablation. For −1 D and −2 D ablations, −0.0034834 (minus 0.0034834) and −0.0069668 (minus 0.0069668) values were used for Z₃, respectively. Initially, the shot patterns were generated to correspond to a VPS value of 0.000056 mm³, which resulted in the ablated lens blanks exhibiting about 70% of the expected result for each of the four dioptric powers. Using a VPS value of 0.000045 mm³ to generate more shot patterns resulted in a diopter change of over 90% of the expected result, as shown in FIG. 9. Given these results, an exact power change is expected to be achievable by utilizing a VPS value of 0.000043 mm³.

The surface profiles of three unablated lens blanks were measured on the interferometer and expressed in terms of Zernike coefficients. Shot patterns for reducing astigmatic aberrations via ablation were generated and applied to the lens blanks. The ablation reduced aberrations to about 1 fringe across the entire 6 mm surface for all three samples.

Two lens blank samples were ablated—after removing a pre-existing astigmatic aberration in a manner discussed above—to test the correction of higher order trefoil aberrations (Z₁₈ for the Zernike numbering scheme used here). Initially, two pure higher order trefoil patterns were created on two lens blank samples by setting Z₁₈ value to either 0.0005 or −0.0005. One sample was ablated with the positive pattern, then the values of Zernike coefficients corresponding to the ablated surface were measured interferometrically. A corrective ablation pattern was then generated based on those coefficients and applied to the surface (several fringes of asymmetrical error remained). A second sample was ablated with the positive pattern, then the negative pattern without removing it from the LADARVision platform. It was observed that the second sample was corrected within 1 fringe. In some cases, the lens blanks were further ablated, after an initial power ablation, to correct surface irregularities. By way of example, in one case the surface error was measured after an initial (−1 D) power ablation, and the surface error was reduced from about 2.8 to about 1.6 microns via subsequent ablations.

### EXAMPLE 4

An Acrysof® Natural lens blank exhibiting pre-existing aberration was ablated by utilizing the aforementioned LADARVision system at 1.35 mJ energy to remove the aberration. The ablation was performed in a 6-mm diameter pupil. The peak-to-valley (P-V) error and Root Mean Square (RMS) error for the lens blank before the ablation were, respectively, 2.42 microns and 0.46 microns. The respective parameters for the lens blank after ablation were 0.74 microns (P-V) and 0.17 microns (RMS), indicating about a three-fold improvement. In at least one other case, the pre-existing aberration was substantially removed.

Those having ordinary skill in the art will appreciate that various changes can be made to above embodiments without departing from the scope of the invention.

What is claimed is:

1. A method of fabricating an intraocular lens (IOL), comprising:
measuring one or more aberrations of a patient's eye,
determining at least one surface profile for a mold wafer based on said measurements,
ablating at least one surface of a mold wafer to impart said profile to that surface, and
utilizing said mold to fabricate an IOL suitable for implantation in said patient's eye.

2. The method of claim 1, wherein said mold wafer is formed of a polymeric material.

3. The method of claim 2, wherein said polymeric material comprises polypropylene.

4. The method of claim 3, wherein said ablating step comprises applying one or more ablative radiation pulses to said surface of the mold wafer with each pulse having a fluence greater than about 100 mJ/cm².

5. The method of claim 1, wherein said IOL is formed of a polymeric material selected from the group consisting of acrylics, hydrogels and silicones.

6. The method of claim 5, wherein said polymeric material comprises AcrySof II.

7. The method of claim 5, wherein each pulse has a fluence in a range of about 100 mJ/cm² to about 800 mJ/cm².

8. The method of claim 1, wherein any of said mold wafer or said IOL is formed of a chromophore material.

9. The method of claim 8, wherein said chromophore material comprises Acrysof Natural or AcrySof II Natural.

10. A method of fabricating an IOL, comprising
measuring one or more aberrations of a patient's eye,
determining one or more surface profiles for an IOL suitable for implantation in said patient's eye,
ablating a substrate formed from a polymeric material so as to fabricate an IOL having said surface profiles.

11. The method of claim 10, wherein said polymeric material can be used as an IOL, such as Acrysof®, hydrogel, or silicone.

12. The method of claim 11, wherein said polymeric material is Acrysof® and said ablating step comprises exposing an Acrysof® surface to an ablative radiation at a fluence in a range of about 10 mJ/cm² to about 600 mJ/cm².

13. The method of claim 11, wherein said polymeric material is AcrySof II.

14. The method of claim 11, wherein said fluence is in a range of about 200 mJ/cm² to about 500 mJ/cm².

15. The method of claim 10, further comprising implanting said IOL in a patient's eye.

16. The method of claim 10, wherein said IOL is formed of a chromophore material.

17. The method of claim 16, wherein said chromophore material comprises Acrysof Natural or AcrySof II Natural.

18. A method of ablating a substrate, comprising
applying a plurality of ablative radiation pulses to a surface of a polymeric substrate so as to impart a desired profile to said surface,
measuring said surface profile to determine one or more surface irregularities, and
applying one or more corrective ablative pulses to said surface so as to reduce said surface irregularities.

19. The method of claim 18, further comprising iteratively measuring said surface profile and applying corrective ablative pulses to the surface until the measured surface irregularities are below a desired threshold.

20. The method of claim 18, wherein said substrate comprises an ophthalmic lens.

21. The method of claim 18, wherein said ophthalmic lens comprises an IOL.

22. The method of claim 18, wherein said substrate comprises a lens blank.

23. The method of claim 18, wherein said substrate comprises a mold wafer.

24. The method of claim 18, wherein said substrate is formed of a soft polymeric material.

25. The method of claim 24, wherein said polymeric material exhibits incubation when exposed to ablative radiation.

26. A method of ablating a substrate, comprising
applying a plurality of shaping ablative radiation pulses according to a pattern to a surface of a polymeric substrate so as to impart a desire profile to said surface,
subsequently, applying one or more corrective ablative pulses according to a predetermined pattern to said surface so as to reduce surface irregularities.

27. The method of claim 26, further comprising determining said pattern of corrective pulses based on a measurement of a surface profile error of another substrate exposed to said shaping ablative pulses.

28. A method of ablating a substrate, comprising
(a) applying a plurality of ablation pulses to a plurality of regions of a surface of the substrate,
(b) subsequent to a selected time period following completion of application of said pluses, applying a plurality of ablation pulses to a plurality of regions of said surface.

29. The method of claim 28, further comprising repeating steps (a) and (b) so as to obtain a desired profile of said surface.

30. The method of claim 28, wherein each of said pulses has a fluence less than about 600 mJ/cm².

31. A method of ablating a substrate, comprising
providing a substrate exhibiting incubation when subjected to ablative radiation,
applying ablative radiation to a surface of said substrate during a plurality of sessions so as to iteratively impart a desired profile to said surface.

32. The method of claim 31, wherein a plurality of ablative pulses are applied to said surface during each session.

33. The method of claim 32, wherein said ablative pulses have a fluence less than a threshold determined based on one or more characteristics of a material from which the substrate is formed.
